(19) United States
(12) Patent Application Publication
(10) Pub. No.: US 2014/0091236 A1
(43) Pub. Date: Apr. 3, 2014

(54) LAMP FIXTURE WITH ONBOARD MEMORY CIRCUIT, AND RELATED LAMP MONITORING SYSTEM

(71) Applicant: ENAQUA, Vista, CA (US)

(72) Inventors: Manoj Kumar Jhawar, San Marcos, CA (US); Gregory Lance Herzog, Cameron Park, CA (US)

(73) Assignee: ENAQUA, Vista, CA (US)

(21) Appl. No.: 14/025,652

(22) Filed: Sep. 12, 2013

Related U.S. Application Data
(60) Provisional application No. 61/707,404, filed on Sep. 28, 2012.
(60) Provisional application No. 61/707,413, filed on Sep. 28, 2012.
(60) Provisional application No. 61/707,423, filed on Sep. 28, 2012.

(51) Int. Cl. G21K 5/00; A61L 2/10

(52) U.S. Cl. CPC G21K 5/00; A61L 2/10
USPC 250/492.1; 250/493.1

(57) ABSTRACT

A system having a host device and a lamp fixture is presented here. The host device includes a lamp receptacle and a host controller coupled to the lamp receptacle. The lamp fixture includes a lamp bulb body, an electrical interface, and a memory circuit coupled to the electrical interface. The electrical interface of the lamp fixture and the lamp receptacle of the host device are physically and electrically compatible with each other. The memory circuit is configured to store operating data associated with the lamp fixture, and the host controller is configured to operate the lamp fixture and to manage data reading and writing operations that involve the memory circuit.

### CROSS-REFERENCE TO RELATED APPLICATION(S)

This application claims the benefit of: U.S. provisional patent application No. 61/707,404, filed Sep. 28, 2012 (titled Intelligent Control Of Lamps In An Ultraviolet Water Disinfection System); U.S. provisional patent application No. 61/707,413, filed Sep. 28, 2012 (titled Inhibiting Open Channel Flow In Water Tubes Of An Ultraviolet Water Disinfection System); and U.S. provisional patent application No. 61/707,423, filed Sep. 28, 2012 (titled Lamp Fixture With Onboard Memory Circuit, And Related Lamp Monitoring System). The content of these provisional applications is incorporated by reference herein.

### TECHNICAL FIELD

Embodiments of the subject matter described herein relate generally to electric lights and lamps. More particularly, embodiments of the subject matter relate to an ultraviolet (UV) lamp fixture having an onboard memory element.

### BACKGROUND

Water treatment systems that use ultraviolet light to disinfect a flow of water are known. A number of ultraviolet-based water treatment systems, arrangements, and architectures have been developed, and such systems utilize the basic disinfecting properties of ultraviolet light. See, for example, the following documents: Anderson, U.S. Pat. No. 6,099,799; Heimer, U.S. Pat. No. 6,303,086; Saccomanno, U.S. Pat. No. 7,169,311; Saccomanno, U.S. Pat. No. 7,498,004; Saccomanno, U.S. Pat. No. 7,534,356; Girodet et al., U.S. Pat. No. 7,947,228; Chang, US 2004/0140269; and Girodet, US 2006/0192135. The relevant content of these documents is incorporated by reference herein.

One type of existing UV water disinfection system employs UV lamps within a flow tank that accommodates open channel water flow. A UV water disinfection system may use a plurality of UV lamps arranged within the flow tank. UV lamp fixtures suitable for use with water disinfection systems are known. Conventional UV lamp fixtures typically employ two or four electrical contact pins for purposes of energizing the lamp filaments, as is well understood. Due to the removable and replaceable nature of such UV lamp fixtures, a technician could remove a UV lamp fixture for some reason, and put it in storage for a period of time. Thereafter, if that UV lamp fixture is placed into service again, it is important to record or otherwise keep track of its new position within the flow tank, its current runtime, etc. Older systems that use conventional lamp fixtures have no convenient way to track and monitor the runtime status, flow tank position, and other information related to the individual UV lamp fixtures (unless a technician manually records data). A simple runtime timer could be used by the system, but the timer would need to be reset or advanced to accommodate the use of replacement lamps.

Accordingly, it would be desirable to have an ability to easily and accurately monitor and record the operating status and other information related to removable lamp fixtures. In addition, it would be desirable to address the shortcomings and deficiencies of conventional lamp fixtures. Furthermore, other desirable features and characteristics will become apparent from the subsequent detailed description and the appended claims, taken in conjunction with the accompanying drawings and the foregoing technical field and background.

### BRIEF SUMMARY

An exemplary embodiment of a lamp fixture includes a lamp bulb body and a memory circuit located external to the lamp bulb body. The memory circuit is configured to store operating data associated with the lamp fixture. In some implementations, the lamp fixture is designed for use in an ultraviolet fluid disinfecting system.

An exemplary embodiment of a system is also presented here. The system includes a host device having a lamp receptacle and a host controller coupled to the lamp receptacle. The system also includes a lamp fixture having a lamp bulb body, an electrical interface, and a memory circuit coupled to the electrical interface. The electrical interface of the lamp fixture and the lamp receptacle of the host device are physically and electrically compatible with each other. The memory circuit is configured to store operating data associated with the lamp fixture, and the host controller is configured to operate the lamp fixture and to manage data reading and writing operations that involve the memory circuit.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a simplified schematic representation of an exemplary embodiment of a water disinfection system;

FIG. 2 is a simplified perspective view of a stage of the system shown in FIG. 1;

FIG. 3 is a simplified schematic representation of a cross-sectional view through a stage of the system depicted in FIG. 1;

FIG. 4 is a perspective view of an exemplary embodiment of a lamp rack suitable for use in the system shown in FIG. 1;

FIG. 5 is a side view of an exemplary embodiment of a lamp fixture having an onboard memory circuit;

FIG. 6 is an end view of the lamp fixture shown in FIG. 5;

FIG. 7 is an exploded perspective view of the connector end of the lamp fixture shown in FIG. 5;

FIG. 8 is a perspective view of an exemplary embodiment of an end cap housing suitable for use with the lamp fixture shown in FIG. 5;

FIG. 9 is an exploded perspective view of the connector end of the lamp fixture shown in FIG. 5, along with a compatible lamp receptacle;

FIG. 10 is a perspective view of a section of the lamp rack shown in FIG. 4;

FIG. 11 is a perspective view of a portion of a lamp fixture configured in accordance with an alternative embodiment;

FIG. 12 is an exploded perspective view of the portion of the lamp fixture shown in FIG. 11;

FIG. 13 is a perspective view of a portion of a lamp fixture configured in accordance with an alternative embodiment;

FIG. 14 is a perspective view of the lamp fixture shown in FIG. 13, mated with a lamp receptacle;

FIG. 15 is an exploded perspective view of the portion of the lamp fixture shown in FIG. 13;

FIG. 16 is an exploded perspective view of a portion of a lamp fixture configured in accordance with an alternative embodiment; and

FIG. 17 is a schematic block diagram representation of a portion of a system that includes lamp fixtures with onboard memory circuits.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. It should be appreciated that block components shown in the figures may be realized by any number of hardware, software, and/or firmware components configured to perform the specified functions.

Certain terminology may be used in the following description for the purpose of reference only, and thus are not intended to be limiting. For example, terms such as “upper”, “lower”, “above”, and “below” refer to directions in the drawings to which reference is made. Terms such as “front”, “back”, “rear”, “side”, “outboard”, and “inboard” describe the orientation and/or location of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second”, and other such numerical terms referring to structures do not imply a sequence or order unless clearly indicated by the context.

For the sake of brevity, conventional techniques related to signal processing, system control, fluid dynamics, ultraviolet-based disinfection, water treatment, and other functional aspects of the systems (and the individual operating components of the systems) may not be described in detail herein. Furthermore, connecting lines shown in any figures contained herein are intended to represent exemplary functional relationships and/or physical couplings between the various elements. It should be noted that many alternative or additional functional relationships or physical connections may be present in an embodiment of the subject matter.

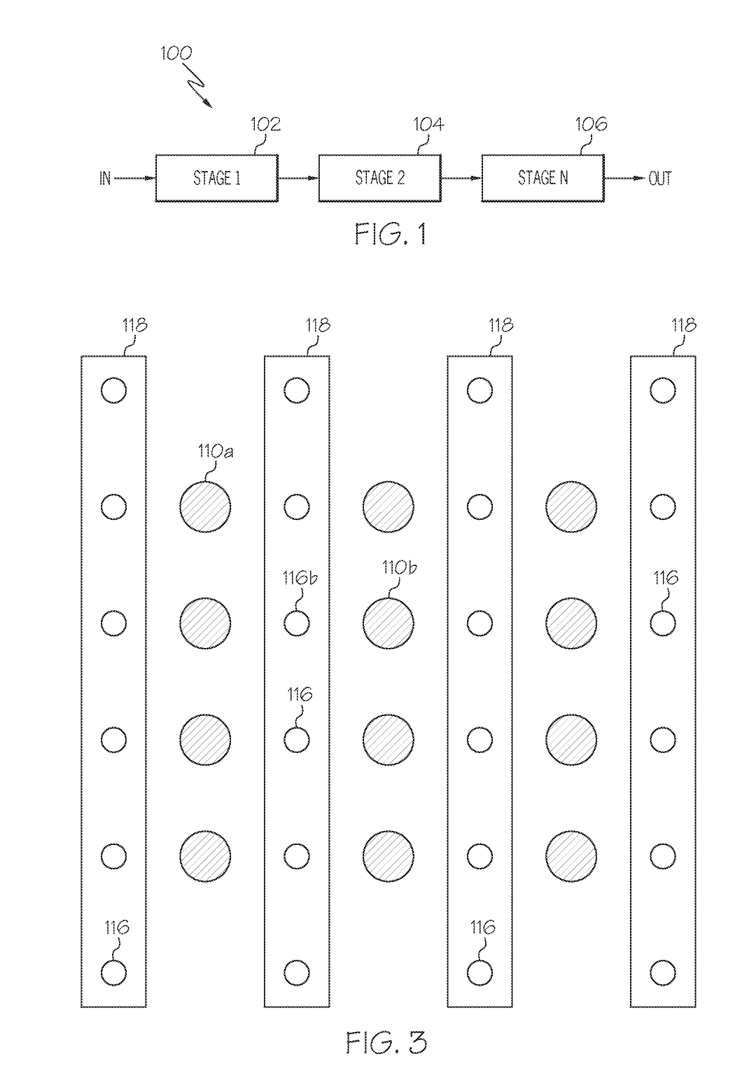

FIG. 1 is a simplified schematic representation of an exemplary embodiment of a water disinfection system 100 that utilizes ultraviolet light technology to disinfect water flowing through the system 100. For the sake of generality, the system 100 is depicted as a multistage embodiment in that the system 100 includes a first stage 102, a second stage 104, and so on. In practice, the system 100 may include only one stage (i.e., the first stage 102 by itself), only two stages (i.e., only the first stage 102 in series with the second stage 104), or any number of stages in series with one another. The first stage 102 receives water to be treated (represented by the “IN” label). The final stage 106 emits treated water (represented by the “OUT” label). In a multistage implementation as depicted in FIG. 1, the output of the first stage 102 serves as the input to the second stage 104, the output of the second stage 104 serves as the input to the final stage 106, and so on. In this regard, water flows through the system 100 in series through the various stages. In practice, each stage of the system 100 may be similarly configured in accordance with the following description. Notably, the system 100 does not utilize an open channel flow scheme. Moreover, the system 100 need not maintain the input and/or output water levels at any defined height. In this regard, the system 100 need not include a weir at the outlet side, or anything functionally equivalent to a weir.

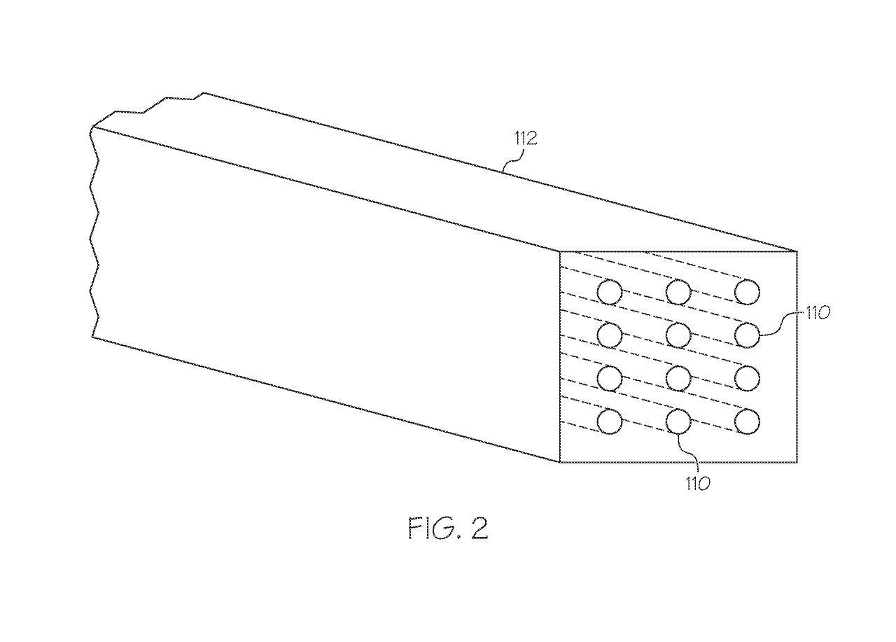

FIG. 2 is a simplified perspective view of a stage 112 of the system 100, and FIG. 3 is a simplified schematic representation of a cross-sectional view through a stage of the system 100. FIG. 2 has been simplified to depict a typical arrangement of water flow tubes 110, which may be arranged along the major longitudinal axis of the stage 112. The number, shape, size, and arrangement of tubes 110 within any given stage may vary from one embodiment to another. For ease of illustration and description, the embodiment depicted in FIG. 2 and FIG. 3 includes twelve tubes 110 arranged in a three-by-four configuration. In a multistage implementation, each tube continues from one stage to another. In other words, each tube 110 in the first stage 102 is coupled to a respective and corresponding tube 110 in the second stage 104, and so on. For example, the tube 110a (depicted in the top left position in FIG. 3) has a corresponding tube 110a in each of the stages and in the same relative position.

Referring to FIG. 3, the stage also includes a plurality of ultraviolet lamp fixtures 116 that are designed to emit ultraviolet radiation to disinfect water as it flows through the tubes 110. In FIG. 3, each of the larger (shaded) circles represents a flow tube 110, and each of the smaller circles represents a lamp fixture 116 (i.e., a UV disinfecting lamp). Although not required in all embodiments, the exemplary implementation illustrated in FIG. 3 has the lamp fixtures 116 configured and arranged in lamp racks 118 that flank the tubes 110. In practice, a stage in the system 100 may have any number of lamp racks 118, and each lamp rack 118 may include any number of lamp fixtures 116. In the illustrated embodiment, the lamp fixtures 116 are substantially aligned with the tubes 110. In this regard, all but two of the rows in FIG. 3 includes three tubes 110 and four lamp fixtures 116. The uppermost and the lowermost rows in FIG. 3 include four lamp fixtures 116, but no tubes 110. Consequently, each tube 110 is surrounded by six neighboring lamp fixtures 116, two of which are immediately adjacent to and flanking the tube 110.

Although not separately shown in FIG. 2, the lamp fixtures 116 in the system 100 are preferably arranged in a longitudinal configuration such that they run substantially parallel to the tubes 110. In alternative embodiments, however, one or more of the lamp fixtures 116 could be perpendicularly arranged relative to the major longitudinal axis of the tubes 110. In yet other embodiments, the lamp fixtures 116 and the tubes 110 need not be orthogonally arranged relative to one another. Moreover, any combination of parallel, perpendicular, and/or non-orthogonal arrangements could be utilized if so desired.

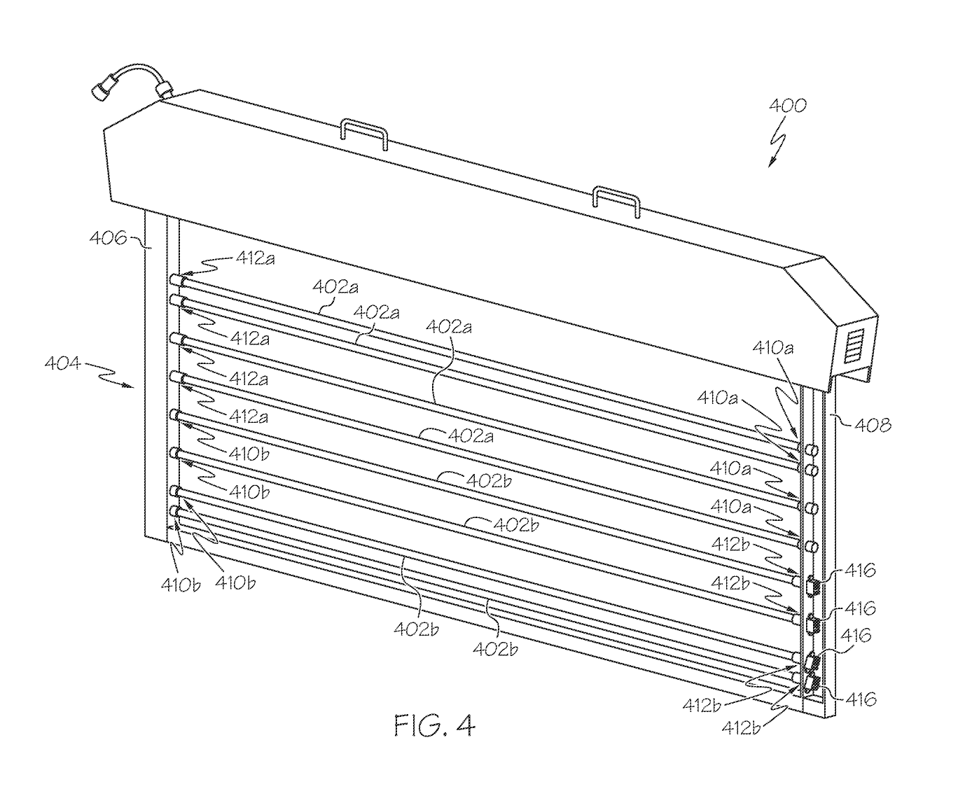

FIG. 4 is a perspective view of an exemplary embodiment of a lamp rack 400 suitable for use in the system 100. Although not always required, the system 100 preferably includes a plurality of identically configured lamp racks 400. For example, each stage in the system 100 may include four lamp racks 400 (see FIG. 3). In certain embodiments, however, the system 100 may utilize any number of differently configured lamp racks.

The illustrated embodiment of the lamp rack 400 includes eight lamp fixtures 402 held within a frame 404. The frame 404 includes a first upright frame section 406 and a second upright frame section 408 that opposes the first upright frame section 406. The ends of the lamp fixtures 402 are supported by the two upright frame sections 406, 408. In this regard, each lamp fixture 402 has a free end 410 and an opposing connector end 412. For this particular embodiment, the four upper lamp fixtures 402a are installed in the opposite direction relative to the four lower lamp fixtures 402b. In other words, the free ends 410a of the upper lamp fixtures 402a and the connector ends 412b of the lower lamp fixtures 402b are supported by the second upright frame section 408, and the free ends 410b of the lower lamp fixtures 402b and the connector ends 412a of the upper lamp fixtures 402a are supported by the first upright frame section 406. It should be appreciated that alternative arrangements and orientations of the lamp fixtures 402 could be utilized if so desired.

A lamp fixture 402 may be installed into the lamp rack 400 as follows. First, the free end 410 of the lamp fixture 402 is inserted into (or otherwise coupled to) a corresponding opening formed in one of the upright frame sections 406, 408. Thereafter, the connector end 412 of the lamp fixture 402 is mated with a corresponding lamp receptacle 416. The lamp receptacles 416 for the upper lamp fixtures 402a are hidden from view in FIG. 4. The lamp receptacles maintain the lamp fixtures 402 in their desired positions along the height of the lamp rack, as shown in FIG. 4.

Each lamp receptacle 416 is physically and electrically compatible with an electrical interface of the lamp fixture 402, which is located at (or incorporated into) the connector end 412 of the lamp fixture 402. Thus, the lamp receptacle 416 provides a physical and electrical connection between the lamp fixture 402 and the host system in which the lamp rack 400 is installed. As described in more detail below, the electrical interface of the lamp fixture 402 may include a number of lamp contacts (e.g., contact pins or conductive sockets) that establish electrical connectivity for purposes of operating the lamp fixture 402.

The host system, such as the system 100, regulates and provides the operating power to each of the lamp fixtures 402, via the respective lamp receptacles 416. In this regard, the host system energizes the filaments of the lamp fixtures 402, which are electrically coupled to at least some of the lamp contacts. Moreover, the host system cooperates with at least one circuit contact to regulate and control the operation of one or more electronic circuits located onboard the lamp fixture 402. In this regard, a lamp fixture 402 may include an onboard memory circuit, a temperature sensor, a light sensor, and/or other electronic components if so desired.

UV lamp fixtures suitable for use with a water disinfection system are known. Certain embodiments of the system 100 described herein utilize lamp technology that relies on two or four contact pins for purposes of energizing the lamp filaments, as is well understood. Ideally, the system 100 or a technician should keep track of the individual lamp fixtures, where the lamp fixtures are located within the system 100 (e.g., which lamp rack and which rack position), and monitor the age, runtime, health, and other operating parameters of the lamp fixtures. Due to the removable and replaceable nature of the lamp fixtures, a technician might remove a lamp fixture for some reason, and put it in storage for a period of time. Thereafter, if that lamp fixture is placed into service again, it is important to record or otherwise keep track of its new position, its current runtime, etc. Older systems that use conventional lamp fixtures have no convenient way to track and monitor the individual lamp fixtures (unless a technician manually enters data into the system 100). A simple runtime timer could be used by the system 100, but the timer would need to be reset or advanced to accommodate the use of replacement lamps.

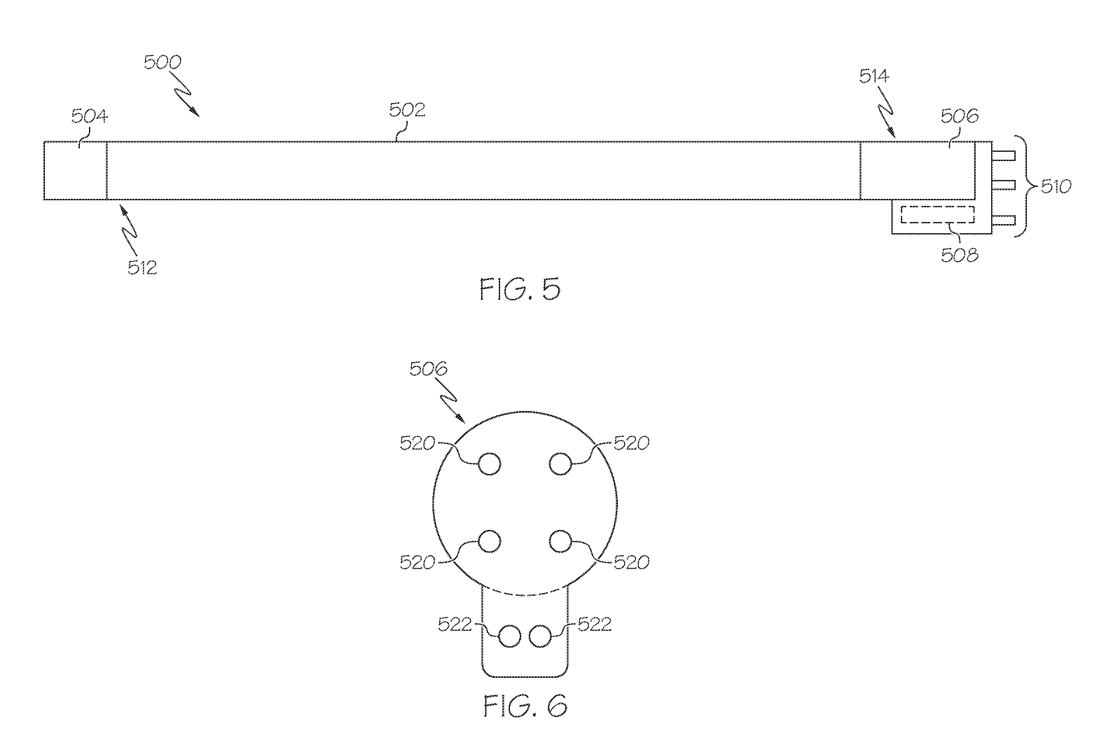

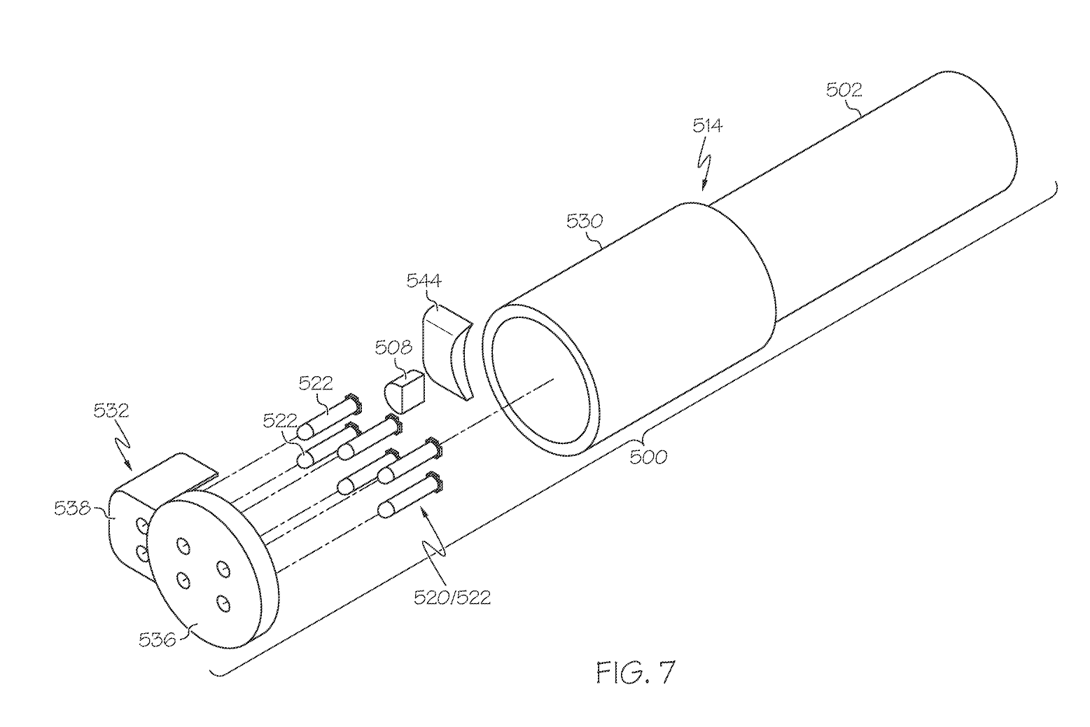

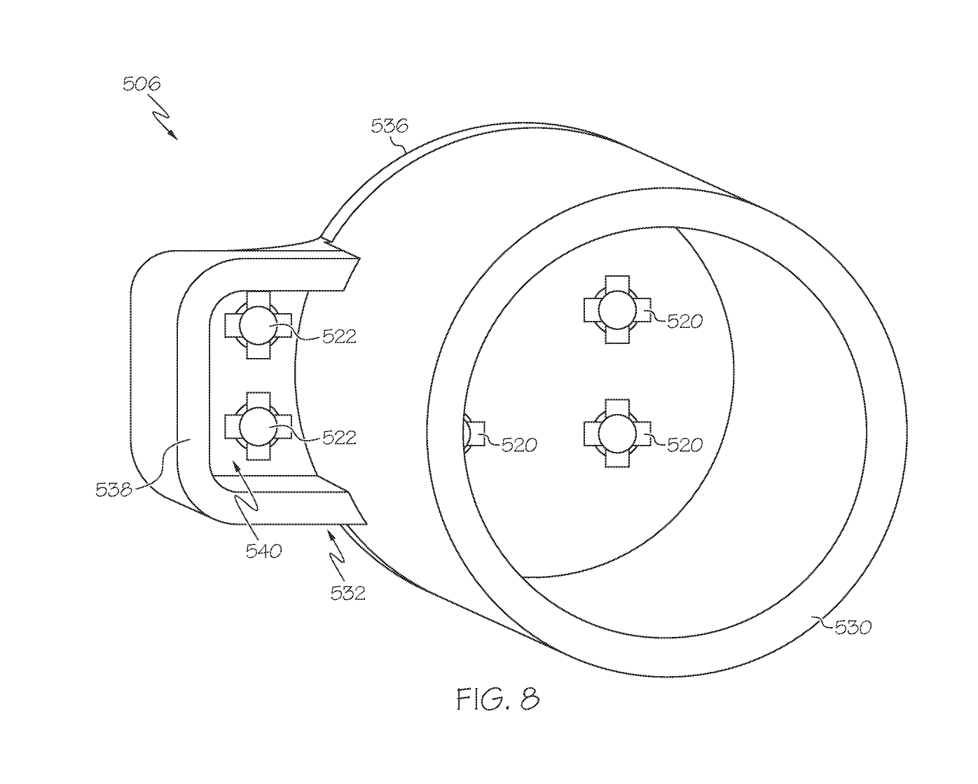

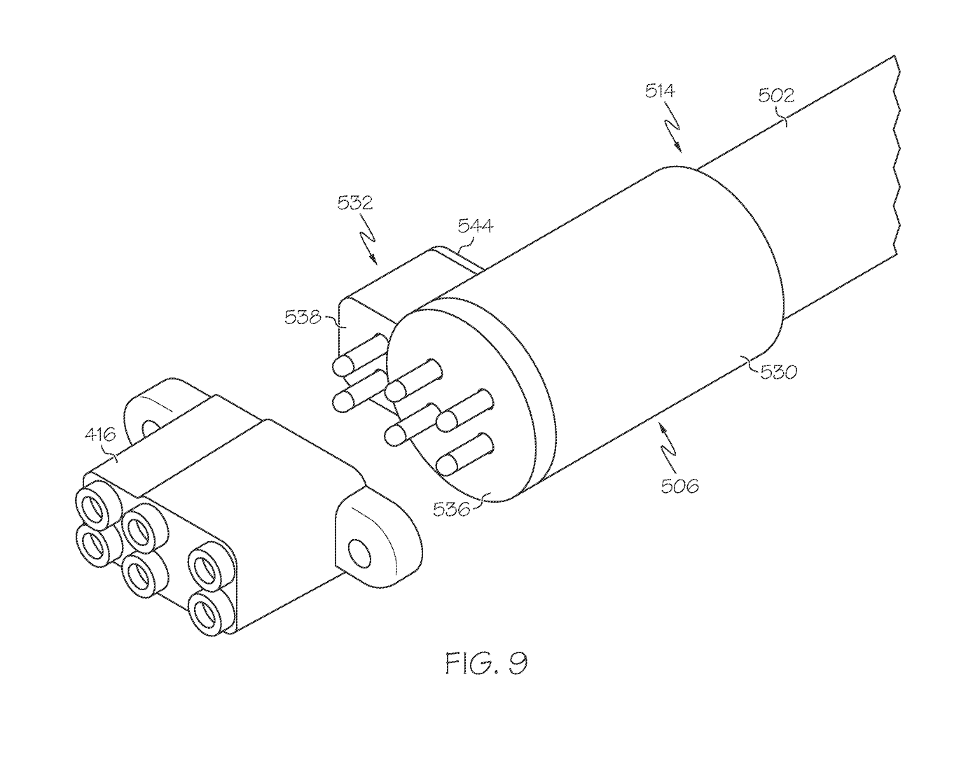

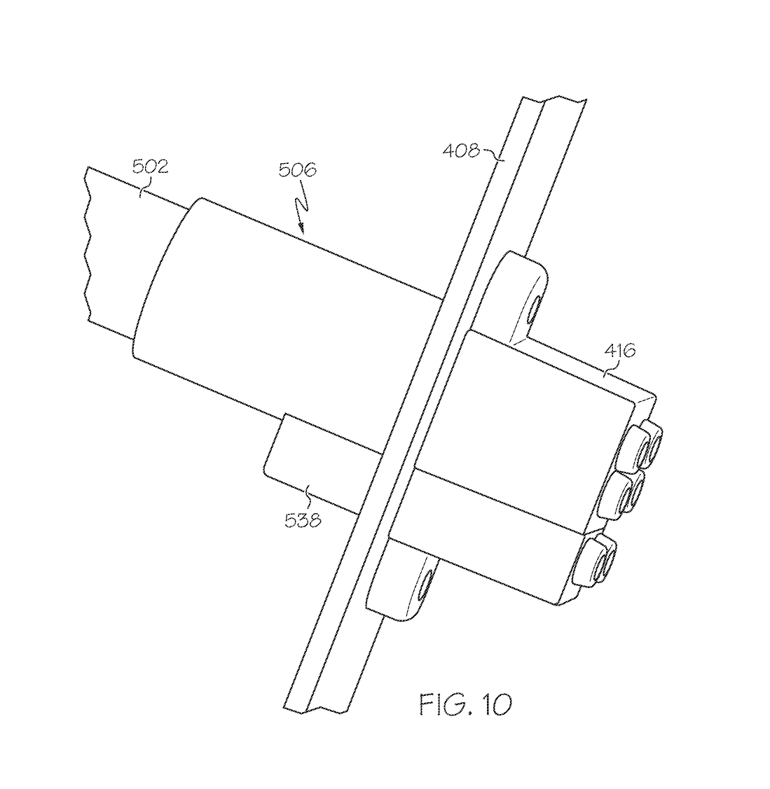

FIGS. 5-9 show various views and components of an exemplary embodiment of a lamp fixture 500 having an onboard memory circuit. More specifically, FIG. 5 is a side view of the lamp fixture 500, FIG. 6 is an end view of the lamp fixture 500, FIG. 7 is an exploded perspective view of the lamp fixture 500, and FIG. 8 is a perspective view of an exemplary embodiment of an end cap housing suitable for use with the lamp fixture 500. FIG. 9 is an exploded perspective view of a connector end of the lamp fixture 500 and a compatible lamp receptacle, and FIG. 10 is a perspective view of a section of the lamp rack 400 shown in FIG. 4. FIG. 10 shows in more detail the manner in which the connector end of the lamp fixture 500 mates with the lamp receptacle.

Referring to FIG. 5, the lamp fixture 500 generally includes, without limitation: a lamp bulb body 502; a first end cap housing 504; a second end cap housing 506; a memory circuit 508; and an electrical interface 510. The lamp fixture 500 may leverage a traditional UV lamp configuration, as is well understood. Accordingly, the lamp bulb body 502, which is typically formed from fused quartz or a glass material, contains the lamp filaments and the gas, chemicals, or molecules that are energized by the filaments. The first end cap housing 504 is secured to a distal end 512 of the lamp bulb body 502. The first end cap housing 504 may be fabricated from a ceramic material, a plastic, a metal material, or the like. Although not depicted in the figures, the first end cap housing 504 may accommodate the routing of wires or electrical conductors from the electrical interface 510 to a filament located within the lamp bulb body 502.

The second end cap housing 506 is coupled to a proximal end 514 of the lamp bulb body 502. The second end cap housing 506 may be fabricated from a ceramic material, a plastic, a metal material, or the like. In some embodiments, the second end cap housing 506 is realized as a one-piece body that supports or is attached to the electrical interface 510. In alternative embodiments, the second end cap housing 506 is constructed as an assembly of two or more pieces that form a body to support or couple with the electrical interface 510. An exemplary two-piece configuration for the second end cap housing 506 is described in more detail below with reference to FIG. 7.

The electrical interface 510 may include an arrangement of electrical contacts protruding from the second end cap housing 506. This particular embodiment includes an arrangement or group of lamp contacts 520 and an arrangement or group of circuit contacts 522, wherein each contact protrudes and extends from the second end cap housing 506. In alternative embodiments, the electrical interface 510 may employ electrically conductive sockets in lieu of some or all of the contacts. The lamp contacts 520 are associated with the filaments of the lamp fixture 500, and the circuit contacts 522 are associated with the memory circuit 508. More specifically, the arrangement of lamp contacts 520 is configured to provide operating power for the filaments of the lamp fixture 500. In this regard, the lamp contacts 520 are utilized to energize the filaments inside the lamp bulb body 502, under the control of the host system, and the circuit contacts 522 are utilized to control read and write operations of the memory circuit 508. For this particular embodiment, four lamp contacts 520 are arranged in accordance with a standardized legacy contact layout, which enables the lamp fixture 500 to be activated, operated, and otherwise controlled in accordance with conventional methodologies. In accordance with certain implementations, the arrangement of circuit contacts 522 includes two circuit contacts 522: a ground contact for the memory circuit 508 and an input-output contact for the memory circuit 508. It should be appreciated that any number of circuit contacts 522 may be used to satisfy the operating requirements of the memory circuit 508.

The illustrated embodiment includes two circuit contacts 522, wherein the arrangement of circuit contacts 522 is offset from the arrangement of lamp contacts 520, as shown in FIG. 6. Moreover, the spacing between the two circuit contacts 522 is different than the spacing between the lamp contacts 520. The inconsistent spacing and offset arrangement of the circuit contacts 522 are desirable to prevent incorrect installation of the lamp fixture 500 into the lamp rack.

In accordance with some embodiments, the horizontal spacing between the lamp contacts 520 (relative to the perspective of FIG. 6) is within the range of about 4 mm to 12 mm, and is about 7 mm for one particular embodiment. In contrast, the horizontal spacing between the circuit contacts 522 is within the range of about 3 mm to 8 mm, and is about 5 mm for one particular embodiment. Although not always required, the horizontal spacing between the circuit contacts 522 is less than the horizontal spacing between the lamp contacts 520 for the embodiment shown in FIG. 6. The vertical spacing between the lamp contacts 520 (relative to the perspective of FIG. 6) is within the range of about 5 mm to 13 mm, and is about 8 mm for one particular embodiment. Moreover, the vertical spacing between the lowermost lamp contacts 520 and the circuit contacts 522 is within the range of about 5 mm to 13 mm, and is about 8 mm for one particular embodiment.

Referring to FIG. 7, the second end cap housing 506 is depicted in a detailed exploded view. The illustrated embodiment of the second end cap housing 506 includes two primary components that are coupled together during assembly of the lamp fixture 500. In this regard, the second end cap housing 506 includes a sleeve section 530 and a cover section 532. The sleeve section 530 may be fabricated from any suitable material such as metal (aluminum), ceramic, or the like. The cover section 532 is preferably fabricated from a dielectric material such as ceramic or plastic, for reasons that will become apparent from the following description. Although this particular embodiment is fabricated from two physically distinct pieces (i.e., the sleeve section 530 and the cover section 532), alternative embodiments may employ a one-piece component that includes the sleeve section and the cover section integrated with one another.

The sleeve section 530 is shaped and sized to accommodate the proximal end 514 of the lamp bulb body 502 (only a portion of which is shown in FIG. 7). During assembly, the proximal end of the lamp bulb body 502 is affixed within the sleeve section 530. The cover section 532 is coupled to the end of the sleeve section 530, as shown in FIG. 8 and FIG. 9. The cover section 532 is fabricated with contact openings that are shaped and sized to accommodate the insertion of the lamp contacts 520 and the circuit contacts 522, which may be realized as electrically conductive pins (as depicted in FIG. 7). The contacts 520, 522 are physically secured within the contact openings using any appropriate technique such that the contacts 520, 522 remain in place and protrude from the cover section 532 as described above. Referring to FIG. 8, the back side of the contacts 520, 522 serve as electrical contact points for the lamp filaments and the memory circuit 508. The lamp filaments and the memory circuit 508 can be physically and electrically coupled to the contacts 520, 522 during fabrication of the lamp fixture 500.

As best shown in FIG. 7 and FIG. 8, the cover section 532 includes a sleeve cap portion 536 and a protruding shell portion 538 that extends from the sleeve cap portion 536. The sleeve cap portion 536 exhibits a generally circular disc shape. Thus, when the cover section 532 is attached to the sleeve section 530, the sleeve cap portion 536 substantially encloses the end of the sleeve section 530. The contact openings for the lamp contacts 520 are formed within the sleeve cap portion 536 such that the lamp contacts 520 are exposed in the interior of the sleeve section 530 (see FIG. 8). The protruding shell portion 538 is located adjacent to the exterior surface of the sleeve section 530, and the protruding shell portion 538 cooperates with the exterior surface of the sleeve section 530 to form a pocket or cavity 540. In this regard, the protruding shell portion 538 exhibits a C-shaped longitudinal cross section, as shown in FIG. 8, wherein the “legs” of the C shape terminate at or near the exterior surface of the sleeve section 530. Notably, the contact openings for the circuit contacts 522 are formed within the protruding shell portion 538 such that the circuit contacts 522 are exposed and accessible within the cavity 540.

The cavity 540 in the second end cap housing 506 is shaped and sized to accommodate the onboard memory circuit 508 (see FIG. 5 and FIG. 8). During assembly of the lamp fixture 500, the contact pins of the memory circuit 508 are electrically and physically connected to the circuit contacts 522, and then the circuit contacts 522 are inserted into the respective contact openings in the protruding shell portion 538. After installation in this manner, the memory circuit 508 resides within the cavity 540. Accordingly, the memory circuit 508 is physically separated from and isolated from the lamp bulb body 502 because the memory circuit 508 is located outside the sleeve section 530. This configuration and arrangement is also desirable to thermally and electrically isolate the memory circuit 508 from the lamp bulb body 502. The lamp fixture 500 may also include a lid 544 for the cavity 540 (the lid 544 is shown in FIG. 7 and FIG. 9). The lid 544 may be secured to the cover section 532 to seal and protect the memory circuit 508.

Moreover, the onboard memory circuit 508 may be encased in epoxy, a silicone material, and/or any suitable material to provide electrical and thermal insulation. In certain embodiments, the insulating material may be provided within the cavity 540 before the lid 544 is affixed. The additional insulation is desirable to ensure that the memory circuit 508 is not adversely affected by the high temperatures, operating voltage, and operating current that is typically associated with normal operation of the lamp fixture 500. In this regard, a typical lamp of this type might experience one to three amps of current and operating temperatures that reach 200-400 degrees F. Notably, the memory circuit 508 does not rely on any of the lamp contacts 520 and does not utilize the voltage or current that is required to operate the filaments of the lamp fixture 500. In other words, the memory circuit 508 is physically, electrically, and thermally isolated from the operating electrical elements of the primary lamp component.

The memory circuit 508 may be realized as an integrated circuit package, a printed circuit board, or the like. In accordance with the exemplary embodiment described here, the memory circuit 508 is realized as a two-conductor integrated circuit device. The two conductors of the memory circuit 508 are electrically coupled to the circuit contacts 522. In certain implementations, the memory circuit 508 is configured in accordance with the 1-WIRE device communications protocol. Consequently, one of the circuit contacts 522 corresponds to ground, and the other circuit contact 522 corresponds to the input-output conductor. Operating voltage for the memory circuit 508 is also provided by the two circuit contacts 522.

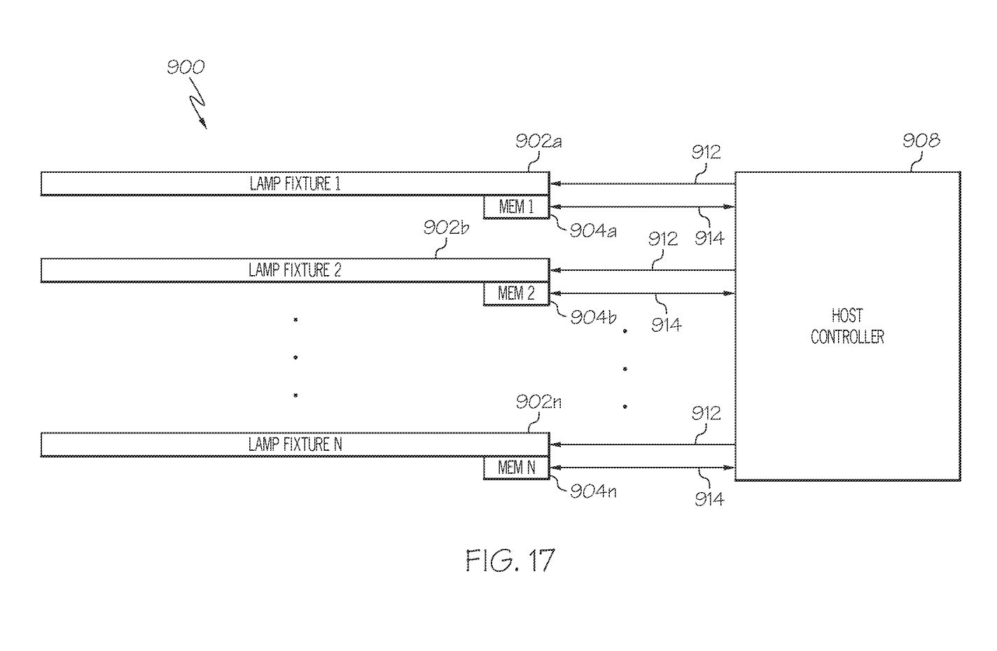

The onboard memory circuit 508 may leverage well-known nonvolatile flash memory technology. In this regard, the memory circuit 508 may include one or more nonvolatile flash memory elements, chips, modules, or the like. As mentioned above, the memory circuit 508 may be configured for compatibility with the 1-WIRE methodology and, therefore, the memory circuit 508 may be realized as a serial memory device that can read and write data in a serial manner. In practice, the memory circuit 508 cooperates with the primary processor or controller of the host system (see FIG. 17) to store operating data that is somehow associated with the lamp fixture 500, to store system data that is associated with the host system, and/or other information as needed. Notably, the nonvolatile nature of the memory circuit 508 is desirable to enable the saved data to remain portable with the lamp fixture 500 (whether or not it remains installed with the host system). Accordingly, the data stored in the memory circuit 508 can be retained at any time, even if the lamp fixture 500 is transported to another system, and even if the lamp fixture 500 is “decommissioned” for a period of time before being reinstalled.

As mentioned above, certain types of lamp-specific data can be written and read by the system as needed. The data stored by the memory circuit 508 may include any or all of the following, without limitation: a minimum temperature generated by the lamp fixture 500; a maximum temperature generated by the lamp fixture 500; the date of manufacture, release, or shipping of the lamp fixture 500; the date of first use of the lamp fixture 500; the total operating runtime of the lamp fixture 500 (as calculated and maintained by the host system); cycling data related to the number of on/off cycles experienced by the lamp fixture 500; the serial number or other identifier of the lamp fixture 500; a manufacturing code of the lamp fixture 500; an original equipment manufacturer (OEM) code of the lamp fixture 500; the most recent or current installation location of the lamp fixture 500 (e.g., a stage or unit identifier, a lamp rack identifier, a column number, a row number, etc.); historical installation locations for the lamp fixture 500; system control parameters for the host system; etc.

In certain scenarios, the operating data stored by the memory circuit 508 may include data that is utilized by the host system for purposes of regulating the operation of the lamp fixture 500. Thus, each lamp fixture 500 may store configuration data, settings, and/or other information that can be accessed and processed by the host system. Similarly, the operating data stored by the memory circuit 508 may include data that is utilized by the host system for purposes of regulating its own operation. Additionally or alternatively, the operating data stored by the memory circuit 508 may include data that is utilized by the host system for purposes of regulating the operation of at least one other lamp fixture in the host system. For example, the operating data stored by the memory circuit 508 may include data that is utilized by the host system for purposes of determining when the lamp fixture 500 should be replaced, maintained, serviced, or the like. Such data may be based on runtime statistics, the date of first use, temperature readings, etc.

In practice, the system 100 may intelligently control the activation of redundant or failover lamps based on the age or health of the primary lamps. To this end, the system 100 can monitor the electrical current drawn by the lamp fixture 500 to determine whether or not the lamp fixture is on or off. When the lamp fixture 500 is active, the system 100 can keep track of its accumulated runtime and write the runtime data to the onboard memory circuit 508. Thus, the lamp fixture 500 will maintain up-to-date information related to its status, condition, and age, and such information will be portable with the lamp fixture 500 such that the lamp fixture 500 can be moved from one location to another, and from one system to another, while still enabling the host system to read the data from the onboard memory circuit 508 when needed.

The lamp monitoring system described here can be used with “binary” lamps (on/off states), lamps with a plurality of discrete output levels, or continuously dimmable lamps. For example, a lamp fixture 500 could store runtime statistics for each operating mode or output level (e.g., low, medium, and high output).

Referring again to FIGS. 6-9, and with further reference to FIG. 10, the protruding shell portion 538 of the second end cap housing 506 results in an asymmetrical end profile for the lamp fixture 500. In this regard, the compatible lamp receptacles 416 exhibit an asymmetric arrangement of electrical sockets to receive the contacts 520, 522 of the lamp fixture 500. An additional benefit of this design is that it makes the lamp fixture 500 easier to align for installation, because the asymmetry results in a self-aligning installation feature. This aspect reduces the likelihood of “forcing” the contacts 520, 522 pins into an incorrect installation orientation, which could result in damage to the lamp fixture 500 and/or the lamp receptacle 416.

### Alternative Embodiments

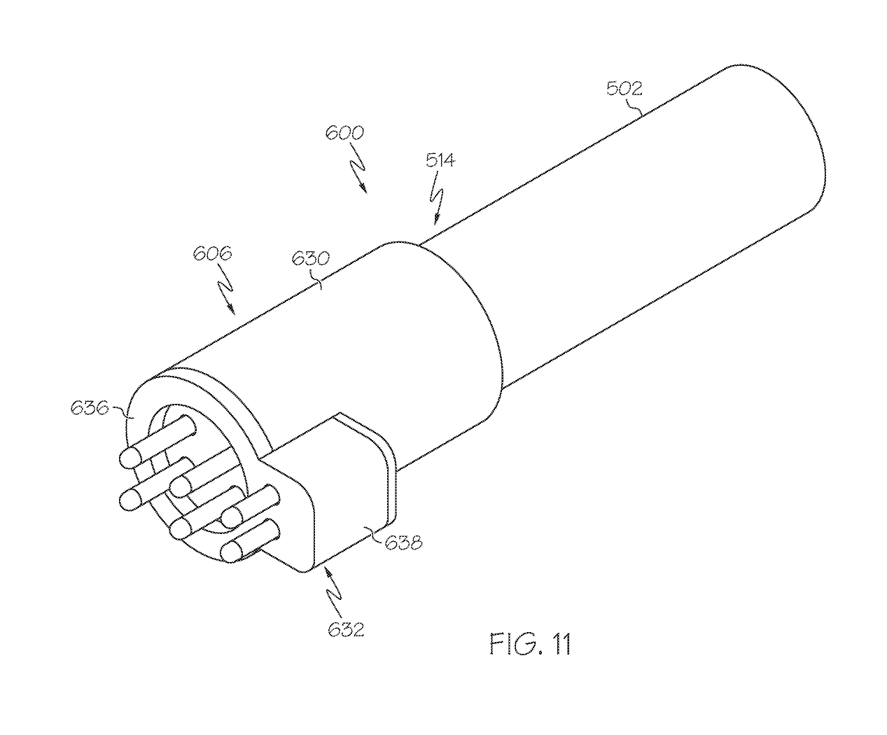

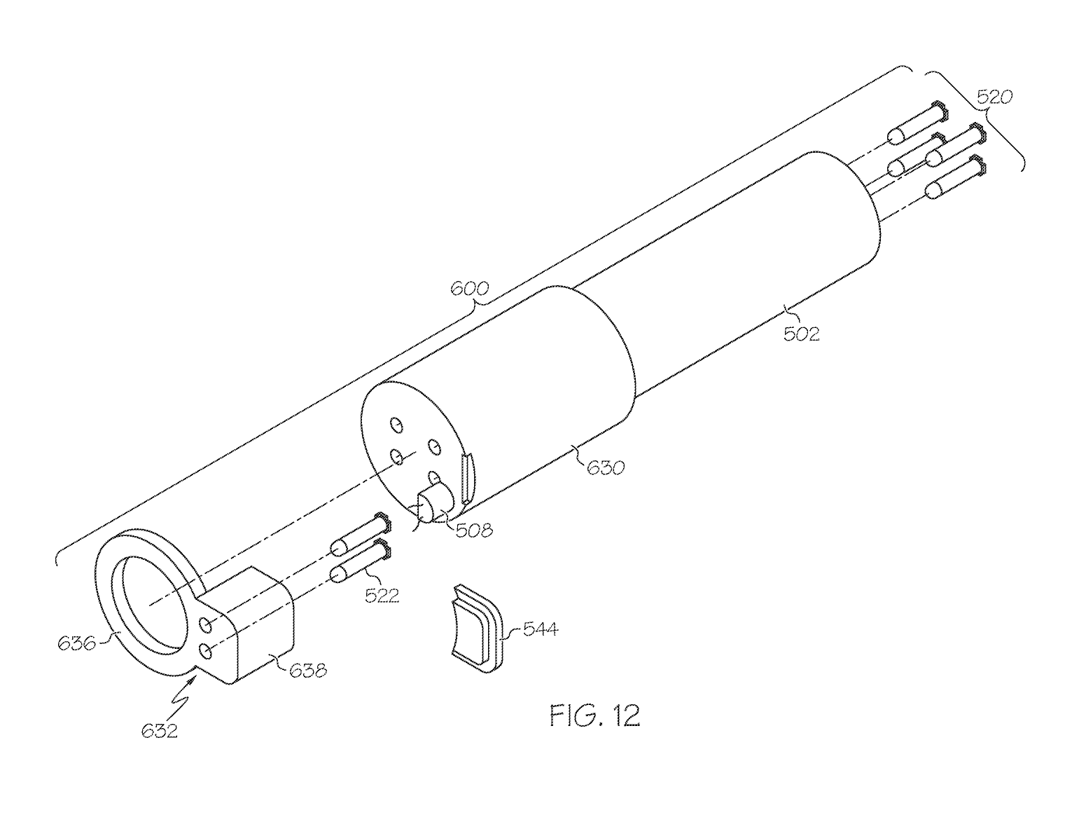

FIGS. 4-10 illustrate a lamp fixture 500 configured in accordance with one exemplary embodiment. It should be appreciated that an electronic circuit can be integrated into a lamp fixture in alternative ways than that described above for the lamp fixture 500. For example, FIG. 11 is a perspective view of a portion of a lamp fixture 600 configured in accordance with an alternative embodiment, and FIG. 12 is an exploded perspective view of the portion of the lamp fixture 600. The lamp fixture 600 has certain characteristics, features, and elements in common with the lamp fixture 500, and such common aspects will not be redundantly described here in the context of the lamp fixture 600.

The lamp fixture 600 includes a second end housing 606 attached to the proximal end 514 of the lamp bulb body 502. The second end housing 606 is realized as a two-part assembly having a capped sleeve section 630 and a cover section 632 coupled to the capped sleeve section 630. In contrast to the sleeve section 530 of the lamp fixture 500 (which resembles an open cylinder), the capped sleeve section 630 is covered at one end, and the contact openings for the lamp contacts 520 are formed in the covered end of the capped sleeve section 630 (see FIG. 12). The cover section 632 includes a ring cap portion 636 and a protruding shell portion 638 that extends from the ring cap portion 636. When the cover section 632 is installed overlying the capped sleeve section 630, the lamp contacts 520 extend through the center of the ring cap portion 636 (see FIG. 11). The memory circuit 508, the circuit contacts 522, and the lid 544 cooperate as described above to secure the memory circuit 508 inside the protruding shell portion 638. After assembly of the lamp fixture 600, the circuit contacts 522 extend through contact openings formed in the end of the protruding shell portion 638 (see FIG. 11).

If so desired, the lamp bulb body 502, the capped sleeve section 630, and the lamp contacts 520 can be fabricated by one vendor, facility, or plant. Thereafter, the cover section 632 (with the memory circuit 508) can be assembled and affixed to the capped sleeve section 630. Thus, fabrication of the lamp fixture 600 can be divided into two primary and discrete steps. Notably, the cover section 632 can be designed for compatibility with an otherwise “standard” or off-the-shelf lamp fixture (having, for example, a four-pin arrangement). Thus, the design of the cover section 632 facilitates the conversion of an standard lamp fixture into one having an integrated component, such as a memory chip as described above.

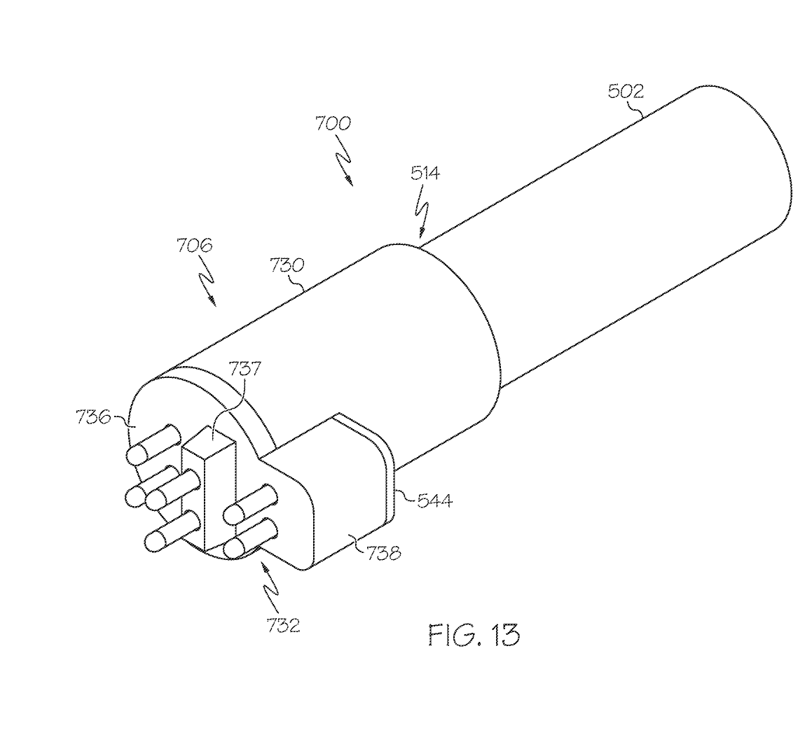

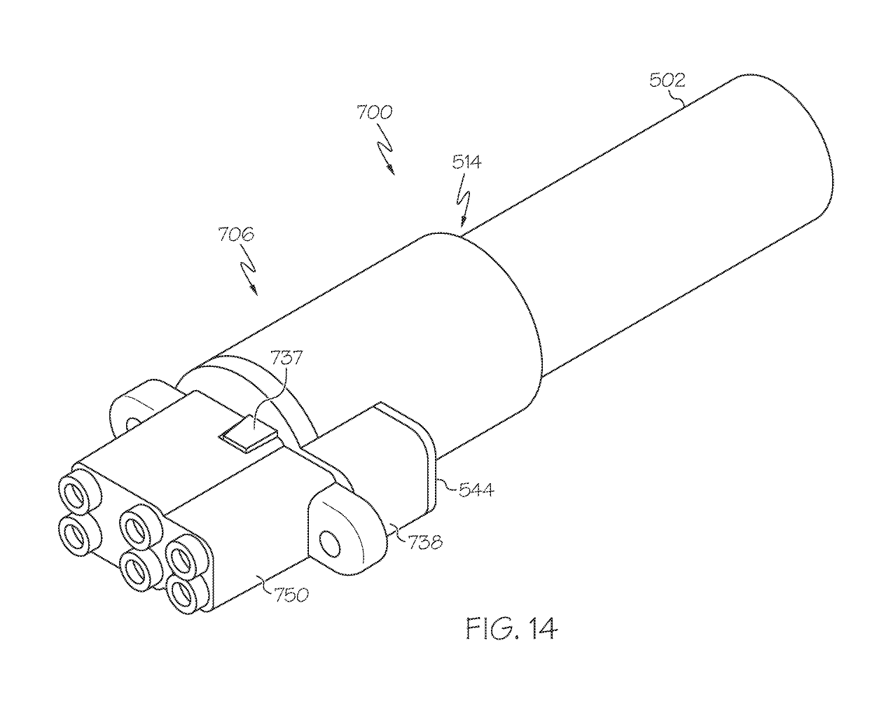

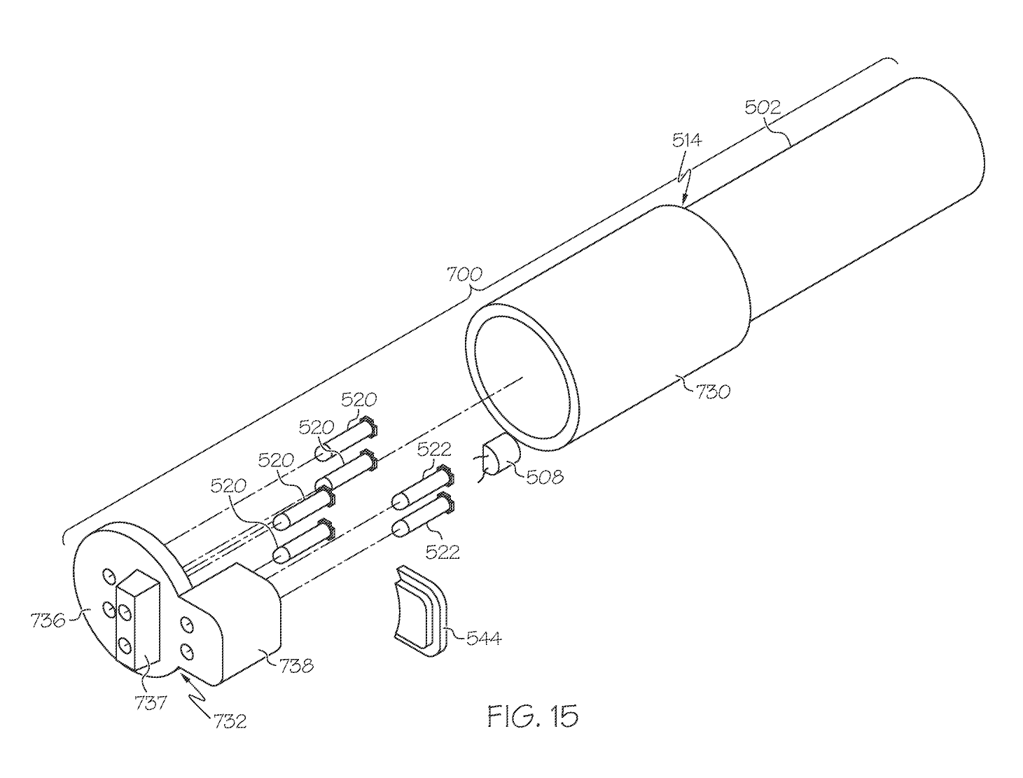

FIG. 13 is a perspective view of a portion of another exemplary embodiment of a lamp fixture 700, FIG. 14 is a perspective view of the lamp fixture 700 mated with a lamp receptacle, and FIG. 15 is an exploded perspective view of lamp fixture 700. The lamp fixture 700 has certain characteristics, features, and elements in common with the lamp fixture 500, and such common aspects will not be redundantly described here in the context of the lamp fixture 700.

The lamp fixture 700 includes a second end housing 706 attached to the proximal end 514 of the lamp bulb body 502. The second end housing 706 is realized as a two-part assembly having a sleeve section 730 and a cover section 732 coupled to the sleeve section 730. The lamp fixture 700 differs from the lamp fixture 500 in that the sleeve cap portion 736 of the cover section 732 includes a contact pedestal 737 protruding therefrom. The contact pedestal 737 includes two contact openings formed therein to accommodate two of the lamp contacts 520. Two other contact openings are formed in the “base” of the sleeve cap portion 736, and these other contact openings accommodate the remaining two lamp contacts 520. The memory circuit 508, the circuit contacts 522, and the lid 544 cooperate as described above to secure the memory circuit 508 inside the protruding shell portion 738. After assembly of the lamp fixture 700, the circuit contacts 522 extend through contact openings formed in the end of the protruding shell portion 738 (see FIG. 13).

As best shown in FIG. 13, the contact pedestal 737 is suitably configured to provide an offset protruding height for two of the lamp contacts 520. Moreover, the contact pedestal 737 may be shaped and sized to mate with a corresponding recess or pocket formed in the lamp receptacle 750 of the host system (see FIG. 14). The offset nature of the two lamp contacts 520, along with the mating features of the contact pedestal 737 and the lamp receptacle 750, provide additional alignment and orientation guidance during installation of the lamp fixture 700 into the lamp rack. These self-aligning features are desirable to ensure that the lamp fixture 700 is properly installed.

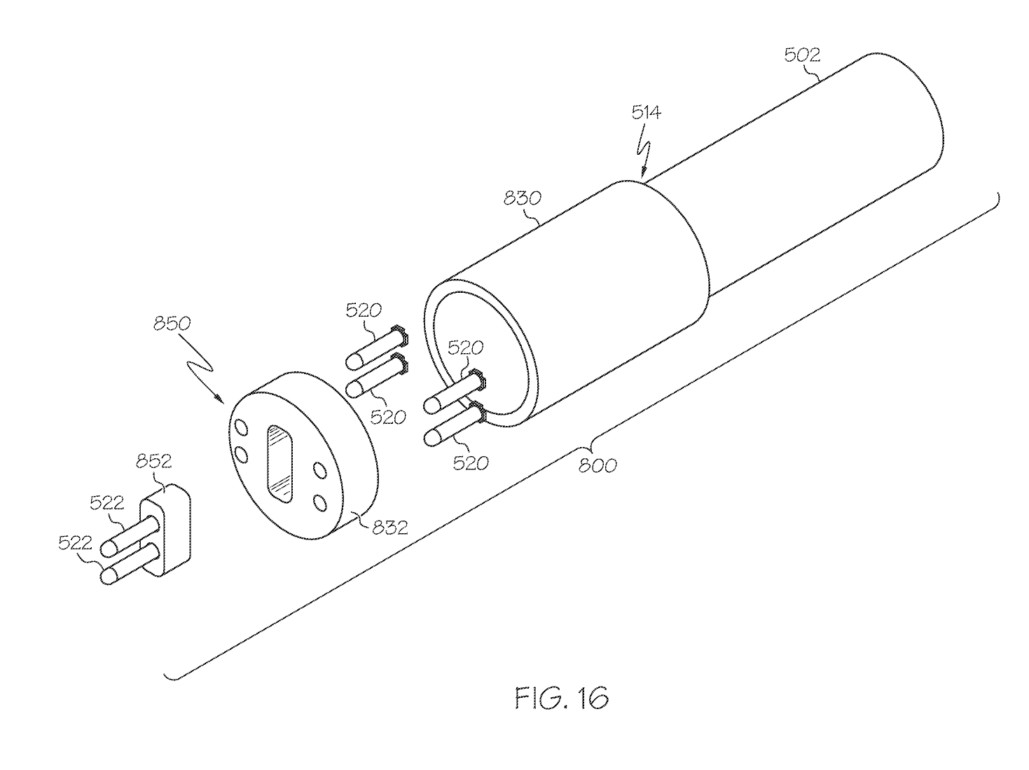

FIG. 16 is an exploded perspective view of a portion of another embodiment of a lamp fixture 800. The lamp fixture 800 has certain characteristics, features, and elements in common with the lamp fixture 500, and such common aspects will not be redundantly described here in the context of the lamp fixture 800.

The lamp fixture 800 includes a second end housing attached to the proximal end 514 of the lamp bulb body 502. The second end housing of the lamp fixture 800 is realized as a two-part assembly having a sleeve section 830 and a cover section 832 coupled to the sleeve section 830. The cover section 832 includes four contact openings to accommodate the lamp contacts 520. Notably, the lamp contacts 520 are generously spaced apart in the lamp fixture 800 (relative to the spacing used in the lamp fixture 500). Accordingly, the illustrated embodiment of the lamp fixture 800 includes a circuit recess 850 formed within the cover section 832. The circuit recess 850 may be used in lieu of a protruding shell portion 538 to accommodate a memory circuit module 852 (or any desired electronic component module, device, circuit, or package).

For this particular embodiment, the memory circuit module 852 includes a memory circuit (hidden from view in FIG. 16) and the corresponding circuit contacts 522. In practice, the memory circuit and the ends of the circuit contacts 522 may be encapsulated in a suitable material to create the memory circuit module 852. Thereafter, the memory circuit module 852 can be secured in the circuit recess 850 using, for example, an adhesive.

If so desired, the lamp bulb body 502, the sleeve section 830, the cover section 832, and the lamp contacts 520 can be fabricated by one vendor, facility, or plant. The memory circuit module 852 can be fabricated independently by a different vendor, facility, or plant if so desired. Thereafter, the memory circuit module 852 can be quickly and easily installed in the circuit recess 850 to complete the fabrication of the lamp fixture 800.

The lamp fixtures described above employ an electrical interface at one end of the lamp bulb body, wherein the electrical interface includes both the lamp contacts 520 and the circuit contacts 522. In alternative embodiments, the electrical interface may be divided such that some contacts are located at the proximal end of the lamp fixture and other contacts are located at the distal end of the lamp fixture. These and other variations in the electrical contact arrangement and configuration are contemplated by this disclosure.

Although the above description focuses on the use of onboard memory circuits, the lamp fixtures described above could be suitably configured for use with any type of electronic component, circuit, device, sensor, transducer, processor, ASIC, or the like. Moreover, although compact integrated circuit packages may be desirable in many applications, the lamp fixtures presented here may also incorporate other circuit technologies, such as printed circuit boards, microstrip, system on a chip (SoC), application-specific integrated circuit (ASIC), or the like. These and other variations in the type and configuration of the onboard component are also contemplated by this disclosure.

Host System

At least one lamp fixture of the type described herein can be deployed in a system that controls the reading and writing of data to the onboard memory circuits. In this regard, FIG. 17 is a simplified schematic block diagram representation of a portion of a system 900 that includes lamp fixtures 902 with onboard memory circuits 904. The system 900 may include a host device having lamp receptacles (not explicitly shown in FIG. 17, but represented by the connecting arrows) and a host controller 908 coupled to the lamp receptacles. As described above with reference to FIG. 4, each lamp fixture 902 has a corresponding lamp receptacle that serves as a physical and electrical connection to the host controller 908. In this regard, the electrical interface 510 of each lamp fixture 902 is physically and electrically compatible with the corresponding interface of the lamp receptacle (see FIG. 5 and the related description). This enables the host controller 908 to regulate and control the reading of data from the onboard memory circuits 904 and/or the writing of data to the onboard memory circuits 904.

In FIG. 17, the arrows 912 are intended to represent the electrical connections and control signals that are used to energize the filaments of the lamp fixtures 902, and the bidirectional arrows 914 are intended to represent the electrical connections, ground voltages, and data signals that are used for purposes of reading and writing data to the memory circuits 904. Notably, the host controller 908 may utilize any suitably written control logic, software, and/or applications to operate the lamp fixtures 902 and to manage data reading and writing operations that involve the memory circuits 904. As mentioned above, certain embodiments leverage the 1-WIRE system architecture and data communication protocol, and the host controller 908 communicates with the memory circuits 904 using 1-WIRE techniques and technologies.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A lamp fixture comprising:
a lamp bulb body; and
a memory circuit located external to the lamp bulb body, wherein the memory circuit is configured to store operating data associated with the lamp fixture.

2. The lamp fixture of claim 1, wherein the operating data stored by the memory circuit comprises data selected from the group consisting of: a minimum temperature generated by the lamp fixture; a maximum temperature generated by the lamp fixture; a date of manufacture of the lamp fixture; a date of first use of the lamp fixture; a total operating runtime of the lamp fixture; cycling data related to the number of on/off cycles experienced by the lamp fixture; a serial number of the lamp fixture; a manufacturing code of the lamp fixture; an original equipment manufacturer (OEM) code of the lamp fixture; a most recent installation location of the lamp fixture within a host system; system control parameters for a host system; and historical installation information of the lamp fixture.

3. The lamp fixture of claim 1, wherein the memory circuit is further configured to store system data associated with a host system compatible with the lamp fixture.

4. The lamp fixture of claim 3, wherein:
the host system comprises an ultraviolet (UV) water disinfection system; and
the lamp fixture comprises a UV disinfecting lamp.

5. The lamp fixture of claim 1, wherein the memory circuit comprises a non-volatile flash memory element.

6. The lamp fixture of claim 1, wherein the memory circuit comprises a serial memory device.

7. The lamp fixture of claim 1, further comprising an end cap housing coupled to the lamp bulb body, wherein the memory circuit is located in the end cap housing.

8. The lamp fixture of claim 7, further comprising:
an arrangement of lamp contacts protruding from the end cap housing, wherein the lamp contacts are associated with filaments of the lamp fixture; and
an arrangement of circuit contacts protruding from the end cap housing, wherein the circuit contacts are associated with the memory circuit.

9. The lamp fixture of claim 8, wherein the arrangement of lamp contacts is compatible with a standardized legacy contact layout.

10. The lamp fixture of claim 8, wherein the arrangement of circuit contacts is offset from the arrangement of lamp contacts.

11. The lamp fixture of claim 8, wherein:
the arrangement of lamp contacts is configured to provide operating power for the filaments of the lamp fixture; and
the arrangement of circuit contacts comprises a ground contact for the memory circuit and an input-output contact for the memory circuit.

12. The lamp fixture of claim 1, wherein the memory circuit is physically isolated from the lamp body.

13. The lamp fixture of claim 1, wherein the memory circuit is thermally insulated from the lamp body.

14. A system comprising:
a host device comprising a lamp receptacle and a host controller coupled to the lamp receptacle; and
a lamp fixture comprising a lamp bulb body, an electrical interface, and a memory circuit coupled to the electrical interface; wherein:
the electrical interface of the lamp fixture and the lamp receptacle of the host device are physically and electrically compatible with each other;
the memory circuit is configured to store operating data associated with the lamp fixture; and
the host controller is configured to operate the lamp fixture and to manage data reading and writing operations that involve the memory circuit.

15. The system of claim 14, wherein:
the host system comprises an ultraviolet (UV) water disinfection system; and
the lamp fixture comprises a UV disinfecting lamp.

16. The system of claim 14, wherein the memory circuit comprises nonvolatile memory that stores the operating data such that the operating data is portable with the lamp fixture.

17. The system of claim 14, wherein the operating data stored by the memory circuit comprises data utilized by the host system for purposes of regulating operation of the lamp fixture.

18. The system of claim 14, wherein the operating data stored by the memory circuit comprises data utilized by the host system for purposes of regulating operation of the host system.

19. The system of claim 14, wherein the operating data stored by the memory circuit is utilized by the host system for purposes of regulating operation of at least one other lamp fixture in the host system.

20. The system of claim 14, wherein the operating data stored by the memory circuit is utilized by the host system for purposes of determining when the lamp fixture should be replaced, maintained, or serviced.
